(19) United States
(12) Patent Application Publication
(10) Pub. No.: US 2025/0292894 A1
(43) Pub. Date: Sep. 18, 2025

(54) SYSTEM AND METHOD FOR TRACKING AND MANAGING SURGICAL TRAY AND MEDICAL ASSETS LOADED THEREON BASED ON DIGITAL IMAGING AND ARTIFICIAL INTELLIGENCE

(71) Applicant: MedGEO Inc., Miami, FL (US)

(72) Inventors: Arun VISWANATH, Barrington, IL (US); Amel NAJJAR, Miami, FL (US)

(21) Appl. No.: 18/606,894

(22) Filed: Mar. 15, 2024

(51) Int. Cl. G16H 40/40; A61B 50/33; G06T 7/00; G16H 30/20; G16H 30/40

(52) U.S. Cl. CPC G16H 40/40; A61B 50/33; G06T 7/0014; G16H 30/20; G16H 30/40; G06T 2207/10004

(57) ABSTRACT

A system and method for tracking and managing a surgical tray and medical assets loaded thereon comprises a user interface for capturing an image of the surgical tray and medical assets loaded thereon at various points of time and locations; a first tangible non-transitory storage medium storing datasets related to the image of the tray and medical assets as captured by the user interface; a second tangible non-transitory storage medium storing datasets related to configuration of surgical trays comprising information on dimensions, colors, and locations of the tray, device identifiers, information on tray identifiers, and information on customers; one or more non-transitory media being connected to the user interface and storing instructions for execution; and a set of computer algorithms that extract information or features from the image and medical assets loaded on the surgical tray received from the one or more non-transitory media.

### TECHNICAL FIELD

The present invention relates to digitized management of surgical trays and medical assets loaded thereon in the clinical settings, particularly, a system for managing surgical trays using digitized imaging of the surgical trays based on artificial intelligence and the method using the system to track and manage surgical trays including medical assets loaded thereon.

### BACKGROUND OF INVENTION

A surgical tray, also known as a surgical instrument tray, a surgical kit, or simply a tray, is a specialized container used in medical and surgical settings to hold and organize various instruments, supplies, implants, and equipment needed for a specific surgical procedure. The instruments refer to the surgical instruments required for specific surgical procedures, such as scalpels, forceps, scissors, retractors, needle holders, and clamps; and the selection of the instruments depends on the type of surgery. The supplies include sutures, surgical drapes, gauze, sponges, sterile gloves, and dressings, which are organized in the surgical tray for easy access during the surgery. The implants refer to medical devices or objects that are surgically placed inside the body to serve specific purposes; they are commonly used in various medical procedures and can be made from a wide range of materials, including metals, plastics, ceramics, and biological materials. The surgical tray usually contains multiple layers, each layer with multiple sub-layers, sections, regions, slots, and sub-slots to hold these medical assets.

There are multiple requirements for preparing and handling the surgical tray. First, the entire surgical tray and the medical assets loaded thereon must be sterilized by autoclaving or ethylene oxide gas sterilization to ensure that it is free from microorganisms that could cause infection. Second, the surgical tray must be organized in a way that makes it easy for surgical staff to access the required items efficiently during the surgical procedure. The instruments and supplies are often arranged systematically to minimize the risk of contamination. Third, the surgical tray is typically labeled with information indicating the type of surgery for which it is intended and the date and time of sterilization to ensure that it is used within their designated shelf life. Fourth, the instruments and supplies within the surgical tray are carefully selected to be compatible with the specific surgical procedure, ensuring that surgeons have the necessary tools at their disposal. Thus, the surgical tray plays a critical role in maintaining the sterility and efficiency of surgical procedures, helps surgical teams work smoothly, and reduces the risk of complications associated with contamination. After the surgery, the medical assets loaded on the surgical tray may be disposed of or sterilized for reuse, depending on hospital protocols and the nature of the items within the tray.

In a healthcare facility, such as a hospital, the Sterile Processing Department (SPD), also known as a Central Sterile Services Department (CSSD) or Central Processing Department (CPD), plays a crucial role in healthcare facilities, such as hospitals and surgical centers. Its primary function is to ensure that medical instruments, equipment, implants, and other items are properly cleaned, sterilized, and prepared for safe and effective use in patient care. The typical workflow of the Sterile Processing Department includes the following processes: (1) receiving and collection; (2) sorting and decontamination; (3) disassembly and inspection; (4) cleaning; (5) sterilization; (6) packaging and storage; (7) distribution and tracking; and (8) quality control and assurance. Sterile processing technicians and other personnel in the department play a crucial role in maintaining the integrity of medical instruments and equipment used in patient care.

Despite various attempts to utilize technology to improve the tracking of surgical assets in a hospital, there are still gaps in tracking handover of assets and trays between vendor, SPD and the departments. Solutions that have tried to address these efficiency issues have either been inadequate in the functionality required to address the nature of the issue or the digital workflows have been burdensome on the users, while lack of tracking has not only serious clinical consequences but also causes errors in the recording, accounting, and billing departments for all the medical professionals involved in the process.

Lack of tracking results in hospitals not being able to track the exact department and the procedure (or patient) that a particular surgical supply was used. This eventually results in hospitals not being able to bill the patient or the respective payor for the assets used at the point of care or surgery. The lack of tracking can also have clinical consequences where hospital or the vendor is not able to track the patient who received a particular implant, for example, in case of a device recall. Vendors also suffer due to inefficiencies in tracking. Due to the high physical handover nature of the workflow between vendors and the hospitals, they are unable to bill hospitals on time for their supplies, the deal with lost supplies and tracking takes up a significant time of a medical sales representative's work schedule.

U.S. Pat. Nos. 10,528,840B2 and 11,367,304B2 belong to the same patent family and disclose a method and system for surgical instrumentation setup and user preferences in an operation room. The method and system described in the patents are based on the captured image of the surgical device in comparison with the desired attributes of the surgical device stored in a digital preference storage using a computer system. However, the patents disclose setting up the surgical device and personnels in one operation occurring in the operation room and do not concern tracking and managing the surgical tray with the loaded medical assets throughout the lifecycle of the surgical tray, particularly, the handovers, movements, assembly, utilization of medical assets, and modification of the surgical tray with changes in the loaded medical assets across the vendor, Sterile Processing department, operation rooms, and other departments in healthcare facilities. Further, the method and system disclosed in the patents do not provide the functionalities of updating the databases and hospital inventory regarding the surgical tray and medical assets loaded thereon in a time sequence and ensuring accurate digital records, integration with patient records in EMRs and/or EHRs, accounting and billing related to the use of the medical assets loaded on the surgical tray.

### SUMMARY OF THE INVENTION

To solve the problems as to lack of efficient tracking and management of the surgical tray and medical assets loaded thereon in the current technology, the present invention provides a digitized tray management system and method with greatly improved efficiency and precision.

The system for tracking and managing a surgical tray in the present invention comprises:
a user interface for capturing an image of the surgical tray and medical assets loaded thereon at one or more points of time and locations;
a first tangible non-transitory storage medium storing one or more datasets related to the image and configuration of the surgical tray and the medical assets loaded thereon at the one or more points of time and locations captured by the user interface;
a second tangible non-transitory storage medium storing one or more datasets related to configuration of surgical trays and the medical assets loaded thereon;
one or more non-transitory media being connected to the user interface and storing instructions that, when executed, causing one or more computing devices to perform steps comprising receiving the image of the surgical tray and the medical assets loaded thereon from the user interface, selecting a computer algorithm for assessing changes in the surgical tray and the medical assets loaded thereon based on a pre-defined logic, and sending the image of the surgical tray and the medical assets loaded thereon received form the user interface to the selected computer algorithm for assessment, and sending information on the surgical tray and the medical assets loaded thereon based on the assessment to the user interface; and
a set of computer algorithms that extract information or features from the image of the surgical tray and the medical assets loaded thereon received from the one or more non-transitory media.

In the present invention, the second tangible non-transitory storage medium comprises a first dataset of information relating to the configuration of surgical trays which include one or more information on dimensions, colors, and locations of the medical assets on the surgical tray and corresponding device identifiers; a second dataset of information on tray identifiers; and a third dataset of information on customers that are associated with the second dataset of information on the tray identifiers.

In the present invention, the system may be installed on a computer, a tablet, or a handheld mobile device. The system may further comprise a build-in camera or an external camera connected to the system, and the camera captures the image of the surgical tray and medical assets loaded thereon.

In the present invention, the set of computer algorithms comprise those based on mean squared error algorithm, structural similarity index, histogram analysis, feature extraction and matching, image registration, or a combination thereof.

The present invention further provides a method for tracking and managing surgical tray and medical assets loaded thereon using the system of the present invention. The method of the present invention comprises the steps of obtaining an image of the surgical tray through the user interface at an initial stage of the surgical tray's lifecycle to obtain an initial reference image, selecting a digital template to be associated with the surgical tray, capturing a series of images of the surgical tray and medical assets loaded thereon during different stages of the lifecycle of the surgical tray, comparing multiple images of the surgical tray and the medical assets loaded thereon, identifying difference in the multiple images and assessing one or more changes in the surgical tray and the medical assets loaded thereon over the different stages of the lifecycle by computer vision analysis, wherein the multiple images of the surgical tray and the medical assets loaded thereon comprise the series of the images captured during the different stages of the lifecycle, the initial reference image, and the digital template, recording and updating the one or more changes in the surgical tray and the medical assets loaded thereon over the different stages of the lifecycle in the datasets stored in the first tangible non-transitory storage medium, and sending an instruction to a user through the user interface based on the one or more changes in the surgical tray and the medical assets loaded thereon, wherein the multiple images of the surgical tray and the medical assets loaded thereon comprise images on a plurality of sections, regions, layers, sub-layers, slots, and sub-slots in the surgical tray, and the one or more changes comprise changes in the physical details and locations of the surgical tray, changes in the medical assets loaded thereon, or both.

The method of the present invention may further comprise the steps of creating the digital template by obtaining a photographic image of the surgical tray, creating a wireframe of the surgical tray based on the photographic image by the computer vision analysis, wherein the computer vision analysis use edge and contouring detection to identify edges and shape of the surgical tray, calculating dimensions of the surgical tray based on a reference object, and organizing the plurality of the sections, regions, layers, sub-layers, slots, and sub-slots in the surgical tray to create the digital template of the surgical tray with corresponding digital sections, regions, layers, sub-layers, slots, and sub-slots.

In the present invention, the method may further comprise the steps of associating one or more sections, regions, slots, or sub-slots of the digital template with a device identifier, wherein the device identifier identifies a device or a medical asset to be fit into the corresponding sections, regions, slots, or sub-slots of the surgical tray associated with the digital template.

In the present invention, the digital template may be modified by scanning the surgical tray and detecting and identifying the medical assets loaded on the surgical tray, and adding and modifying the digital template with the identified medical assets loaded on the surgical tray. The graphical user interface enables a user to capture the image of a surgical tray and create or modify an existing template based on the captured image.

Furthermore, the digital template may be selected based on a tray identifier provided on or associated with the surgical tray or from the second tangible non-transitory storage medium comprising pre-determined digital templates. When a digital template is selected from the database, the method of the present invention may further comprise the steps of analyzing the initial reference image of the surgical tray by the computer vision analysis, obtaining and displaying a plurality of the pre-determined digital templates related to the surgical tray from the database on the user interface, and selecting the digital template associated with the surgical tray among the plurality of the displayed digital templates by the user through the user interface.

In the present invention, the method may further comprise the step of associating a digital signature of the user with the changes in the surgical tray and the medical assets loaded thereon.

In the present invention, the method may comprise sending the following instructions based on the assessed changes in the surgical tray itself or the medical assets filled in the surgical tray, or both. When the changes in the surgical tray comprise a misplaced medical asset, the instruction is to correct the misplaced medical asset. When the changes in the surgical tray comprise changes in quantities of the medical assets in the surgical tray, the instruction may be to update accounting for the medical assets and billing in accordance with the changes, or to replenish the supply for the medical assets, or both.

In the present invention, the method may further comprise detecting a triggering event for updating the one or more changes of the surgical tray in the one or more datasets stored in the first tangible non-transitory storage medium, and updating the one or more datasets stored in the first tangible non-transitory storage medium, wherein the triggering event is a hand gesture or movement by the user near the surgical tray, set by a pre-configured timer, or an action of taking a photo or scanning of the surgical tray by the user.

In the present invention, the method may further comprise arranging the multiple images of the surgical tray in the different stages of the lifecycle in a time sequence, and displaying the one or more changes associated with the different stages of the lifecycle through the user interface, wherein the one or more changes comprise usages and disposition of the medical assets and users associated with the one or more changes.

Further, the method of the present invention may comprise the steps of identifying a fiduciary indicator on the surgical tray in the different stages of the lifecycle, and recording the one or more changes associated with the fiduciary indicator.

The present invention is particularly useful in tracking and managing implants as the medical assets loaded on the surgical tray.

### BRIEF DESCRIPTION OF THE DRAWINGS

FIG. 1 is a diagram showing the structure of the system for tracking and managing a surgical tray of the present invention.

FIG. 2A shows the structure of a surgical tray with multiple layers and sub-trays; FIG. 2B shows the hierarchy in the structure of the surgical tray.

FIG. 3A shows detailed structure in one layer of the surgical tray containing regions, slots, and sub slots; FIG. 3B shows detailed structure in one layer of the surgical tray with medical assets loaded in some slots.

FIG. 4A shows the first arrangement for capturing the image of the surgical tray in the present invention; FIG. 4B shows the second arrangement for capturing the image of the surgical tray in the present invention; and FIG. 4C shows the third arrangement for capturing the image of the surgical tray in the present invention.

FIG. 5A is a photographic image of a layer of the surgical tray captured in the present invention; and FIG. 5B is the digital template of the same layer created by the method of the present invention.

FIG. 6 shows the history of the locations of multiple surgical trays in a time sequence in one embodiment of the present invention.

Reference numbers used in the figures refer to the following structural components: 1—system of the present invention; 10—surgical tray; 11—layer of a surgical tray; 12—sub-tray; 13—region of a surgical tray; 14—slot; 15—sub slot; 16—items vertically stacked in a slot; 17—item placed in a slot; 21—camera; 22—camera mount; 23—tablet mount; 30—computer; 31—tablet computer; 32—mobile device;
100—user interface (GUI); 200—first database; 300—second database; 301—dataset on configuration of the surgical tray; 302—dataset on tray identifier; 303—dataset on customer information; 400—non-transitory medium; 500—computer algorithm (means for computer vision analysis); 501—mean squared error algorithm; 502—structural similarity index; 503—histogram analysis, 504—feature extraction and matching, 505—image registration.

### DETAILED DESCRIPTION OF THE INVENTION AND EMBODIMENTS

The present invention is further explained in conjunction with the drawings and examples, but the scope of protection of the present invention shall not be limited thereby.

The system and method of the present invention can be used in various clinical settings to digitize surgical trays during the course of the usage and movement of the surgical tray with medical assets loaded thereon within various departments at a hospital or healthcare facility. Specifically, the system and method of the present invention provide accurate count of asset utilization of a surgical tray or a set of surgical trays. The level of optimization and accuracy resulting due to the present invention enables operating rooms to have the exact set of assets or parts required to perform a procedure and allows timely replenishment of inventory.

In the present invention, a “digitized tray management” or “DTM” is the digital version of a tray and its medical assets loaded thereon recorded during the flow of the tray and its medical assets loaded thereon between the Sterile Processing Department (SPD), clinical care areas such as the Operating Rooms (OR), and vendor. The digitized tray management is a computerized repository of tray information created and stored in accordance with the system and method of the present invention. The digitized tray management provides a comprehensive and accurate record of the content of the tray, its usage, access records and history of its handover between various departments in a hospital and the vendor.

In the present invention, a “medical procedure” or “medical procedures” refer to any practice of a health practitioner that involves a combination of special skills or abilities and may require one or more medical devices or supplies. A medical procedure may be surgical or non-surgical, preventive, rehabilitating, diagnostic, therapeutic, anesthesia, or propaedeutic.

In the present invention, a “healthcare facility” or “healthcare facilities” refer to places and entities that provide medical care, including but not limited to, hospitals, medical offices, medical centers, urgent care centers, and nursing centers or homes.

In the present invention, a “medical device” or “medical devices” refer to the same term as defined under Section 201(h) of the Food, Drug & Cosmetic Act in the United States and regulated by the FDA. The medical device can range from simple tongue depressors and bedpans to complex programmable pacemakers, and closed loop artificial pancreas systems. Additionally, medical devices include in vitro diagnostic (IVD) products, such as reagents, test kits, and blood glucose meters. Certain radiation-emitting electronic products that have a medical use or make medical claims are also considered medical devices. Examples of these include diagnostic ultrasound products, x-ray machines and medical lasers. Medical devices includes consumable, implantable, and explantable medical devices in the present invention.

In the present invention, “medical supplies” or “surgical supplies” refer to any other tool or material that are not regulated as medical devices by the FDA but nevertheless used during or in connection with a medical procedure.

In the present invention, “medical assets” or “assets” refer to products that include medical equipment, implants, consumables, and other medical devices that are used during the medical procedure. Instruments, medical supplies, and implants are referred to collectively as medical assets or assets in the present invention.

Particularly, in the present invention, an “implant” or “implants” is a medical device or material that is surgically placed inside the body to replace, support, or enhance a biological structure or function. The implants are implantable medical assets that can be made from a variety of materials such as metals, ceramics, plastics, or biological tissues. They are designed to integrate with the body's tissues or organs, either temporarily or permanently, to achieve a specific therapeutic or corrective outcome. These include plates, screws, rods and several other devices that can be made from a wide range of materials, including metals, plastics, ceramics, and biological materials. Further, in order to facilitate surgical implantation of implants, both implantable medical assets and associated products are filled in the surgical trays. The associated products or medical assets include drill bits, screw drivers, wires, instruments and cutting guides that facilitate the implantation of the implantable products which may also be organized in the surgical tray. Furthermore, the surgical trays may be filled with supplies which are medical assets that facilitate the surgical placements of the implantable hardware, including the supplies of surgical drapes, gauze, dressings and other associated products, to facilitate the surgical surgical procedure. For the purpose of the application, the implants include all of the implantable medical assets, associated products, and other supplies.

In the present invention, a “user” includes one or more clinical users and vendor users. The “clinical user” is a user of the present invention that is usually associated with a healthcare facility that uses the present invention to track and manage the surgical tray and its medical assets loaded thereon. In contrast, a “vendor user,” also called a “rep user,” is a user of the present invention that is a representative from the vendor or suppliers of the medical assets.

In the present invention, an “interface” or a “user interface” refers to a connection between software applications, application and database, or application and user. The graphic user interface (GUI) particularly refers to the interface using graphics.

In the present invention, “electronic records (ER)” include electronic medical records (EMR), electronic health records (EHR), and enterprise resource planning records (ERPs), that are commonly used in the departments in a healthcare facility.

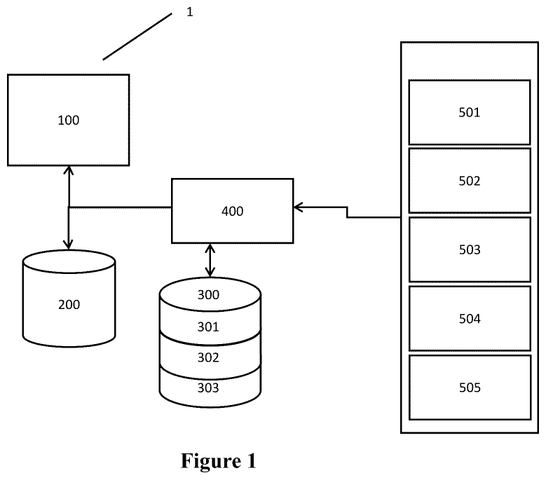

As shown in FIG. 1, the system 1 of the present invention comprises a user interface 100 for capturing an image and medical assets loaded thereon of a surgical tray at one or more points of time and locations; a first tangible non-transitory storage medium or database 200 storing one or more datasets related to the image and configurations of the surgical tray and the medical assets loaded on the surgical tray at the one or more points of time and locations captured by the user interface 100; a second tangible non-transitory storage medium or database 300 storing one or more datasets (301, 302, 303) related to configuration of surgical trays; a non-transitory medium (in this case, a request manager) 400 being connected to the user interface 100 and storing instruction that, when executed, causing one or more computing devices to perform steps comprising receiving the image and medical assets loaded on the surgical tray from the user interface 100, selecting a computer algorithm for assessing changes in physical details, locations, and medical assets loaded on the surgical tray based on a pre-defined logic, where the pre-defined logic maps specific algorithms to surgical tray types, for examples, mapping the color or size of the tray; the asset types such as metallic assets including screws, and sending the image and medical assets loaded on the surgical tray received form the user interface 100 to the selected computer algorithm for assessment, and sending information on the medical assets loaded on the surgical tray based on the assessment to the user interface; and a set of computer algorithms 500 that extract information or features from the image and medical assets loaded on the surgical tray received from the first non-transitory medium.

As shown in FIG. 1, the second tangible non-transitory storage medium or data repository 300 comprises a first dataset 301 of information relating to the configuration of surgical trays comprising one or more information on dimensions, colors, and locations of assets on the surgical tray, and device identifiers for the medical assets that may be loaded onto the surgical tray, a second dataset 302 of information on tray identifiers, and a third dataset 303 of information on customers, wherein the information on customers are associated with the second dataset of information on the tray identifiers.

The system 1 of the present invention may be divided into a first subsystem that provides digital tray management services (100, 200, 400, and 500) and a second subsystem that contains the data repository (300). The digital tray management services subsystem comprises a set of computer application services that run either on a server in a data center or in cloud which is deployed on a cloud service provider such as Amazon AWS or Microsoft Azure, and is made up of the user interface 100, first database 200, request manager 400, and a set of computer algorithm 500, and can be accessed through the computer application or using a web browser on various supported clients.

The user interface (GUI) 100 of the subsystem can run on the user's devices including computer workstations, laptops, tablets, and mobile devices. The devices may use the inbuild camera or external camera to capture images and the medical assets loaded thereon of a tray.

The first database 200 comprises datasets of information on tray images and configuration, and the medical assets loaded thereon of the tray, including medical assets filled in the slots and sub slots in the tray. Tray images are set of pictures of the tray sent from the user interface for computer vision analysis. Tray configuration is physical and content details of a tray that help the computer vision algorithms to evaluate the changes to a tray. The datasets may include information like the dimension of the tray, colors, location of various assets in the tray, and device identifiers for the medical assets.

The non-transitory medium 400, in this case a request manager, is a software component that the user interface connects to, and sends tray identifiers and tray images. The job of the Request Manager is to take the information from the user interface and chooses the appropriate computer vision algorithm 500 based on predefined logic, such as like tray type and tray size, to identify the parts that have been added or removed from the tray.

The set of computer vision algorithms 500 is a component that includes a set of mathematical and computational techniques designed to extract meaningful information or features from visual data. The extracted information in this case are assets that have been added or removed from a tray, location where assets are added or removed and the respective asset identifiers. The set of computer algorithms 500 are based on mean squared error algorithm 501, structural similarity index 502, histogram analysis 503, feature extraction and matching 504, image registration 505, or a combination thereof.

Computer vision is a field of artificial intelligence (AI) and computer science that focuses on enabling computers and machines to interpret and understand visual information from the world, much like the human visual system. It involves developing algorithms and techniques that allow computers to extract meaningful insights, recognize objects, patterns, and gestures, and make decisions based on visual data from images and videos. The set of computer algorithms 501 to 505 used in the computer vision analysis are known in the art. Analysis of computer vision methods for counting surgical instruments is described, for example, in an article by Chavez et al., “Analysis of Computer Vision Methods for Counting Surgical Instruments,” Surgical Innovation, 2021, Vol. 28 (3), pp. 392-393, which is incorporated herein by reference.

Particularly, Mean Squared Error (MSE) is a computer vision algorithm that measures the average squared difference between the pixel values of two images. Structural Similarity Index (SSI) is used in the field of image and video processing, as well as computer vision, to assess the perceived quality of an image or video compared to a reference image or video. It is designed to measure the structural similarity between two images or video frames by considering various aspects of human perception. In the field of computer vision, histograms analysis uses image histograms as useful tools for thresholding. As the information contained in the graph is a representation of pixel distribution as a function of tonal variation, image histograms are analyzed for peaks and/or valleys in histograms analysis algorithm. Feature extraction and matching in computer vision refers to the computer algorithm where features detected in each image can be matched across multiple images to establish corresponding features such as corresponding points; the algorithm is based on comparing and analyzing point correspondences between the reference image and the target image. Image registration in computer vision refers to the process of spatially aligning two or more image datasets of the same scene taken at different times, from different viewpoints, and/or by different sensors.

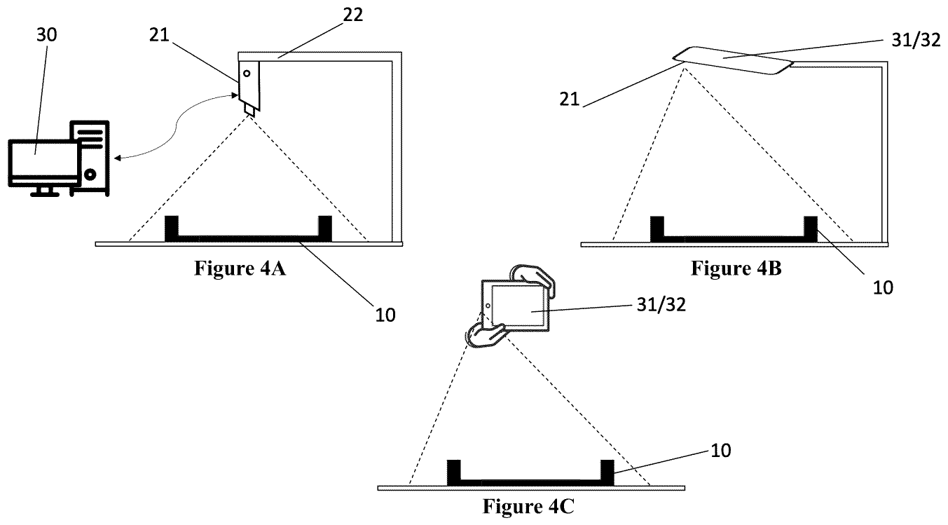

The second subsystem refers to the second dataset 300, which is a centralized data repository that stores tray configurations, list of trays and their identifiers linked to respective customers (like hospitals and medical device vendors). As shown in FIGS. 4A to 4C, the system 1 may have an associated camera to enable scanning or capture of containers or trays during the workflow described in the overview. The system may also have a database of tray or container templates.

In the present invention, the system 1 may be installed on a computer, a tablet, or a handheld mobile device and further comprises a build-in or external camera connected thereto, and the camera captures the image and medical assets loaded on the surgical tray.

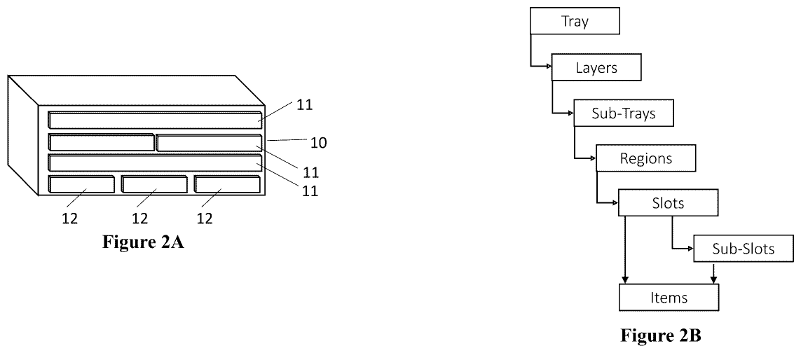

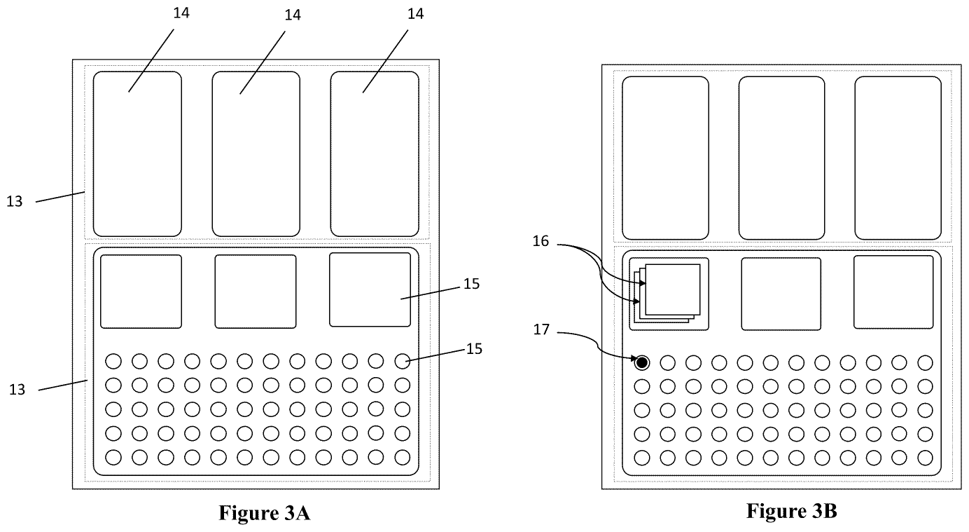

FIGS. 2A, 2B, 3A, and 3B show the structure and organization of a surgical tray for which the method of the present invention creates the digital template and tracking and managing its configuration and medical assets loaded thereon.

As shown in FIG. 2A, surgical tray 10 comprises a plurality of layers 11. In each layer, it may contain multiple sub-trays 12. As shown in FIG. 2B, the hierarchy of the structural components in the surgical tray starts with a tray or a surgical tray, which is a sterile platform used to organize and hold surgical instruments during medical procedures, which typically consists of sub-trays and slots designed for the systematic arrangement of instruments and implants. A typical surgical tray may have multiple sub-trays arranged in layers of efficient organization. A sub-tray is a unit that holds multiple instruments and implants, which has multiple regions within the sub-tray to organize items for efficient access. A slot within a sub-tray region serves the purpose of holding a particular item or type of item. A slot may have one or more sub-slots, and these sub-slots serve the purpose of holding a particular item or type of item.

For simplicity, the various layers, sub-layers, regions, slots, sub-slots, sub-trays where the medical assets are filled are referred to collectively as the surgical tray, and each of the various layers, sub-layers, regions, slots, sub-slots, sub-trays in the surgical tray is referred to as a section of the surgical tray in the present invention. An item refers to surgical instruments or surgical consumables or implants used during the operating procedure.

As shown in FIG. 3A, in one layer of the tray, it may contain a plurality of regions 13, and each region may contain slots 14 or sub slots 15. FIG. 3A shows that the tray is not loaded with any medical assets. FIG. 3B shows that medical assets 16 are stacked vertically in a sub slot or one single medical asset 17 is fit into one sub slot.

As shown in FIGS. 4A to 4C, in the present invention, images are captured during the initial stage and various time and location points as well as through the entire lifecycle by different arrangements.

In the arrangement as shown in FIG. 4A, the system of the present invention is accessed using a computer or a laptop 30. The computer or laptop 30 is attached to an external camera 21 that can be held on a camera mount 22 attached to a table. The surgical tray 10 is placed directly below the camera.

In the arrangement as shown in FIG. 4B, instead of a computer or a laptop, a tablet 31 or a mobile device 32 is being used for the application in the system of the present invention. Also, in this case, a built-in camera 21 is used instead of an external camera, and the tablet 31 or mobile device 32 is mounted on a tablet mount 23.

In the arrangement as shown in FIG. 4C, the user holds the tablet 31 or mobile device 32 that is connected to the computer application in the system, instead of using a mount 23.

The present invention creates a digital template of a tray based on a photographic image of a physical tray captured through various arrangements. The present invention has a mechanism to create wireframe templates based on an image of a physical tray. The system and method of the present invention use a computer vision mechanisms like edge detection and contouring to identify the edge and shape of the tray. In addition, it uses the same computer vison mechanisms to identify various tray sections.

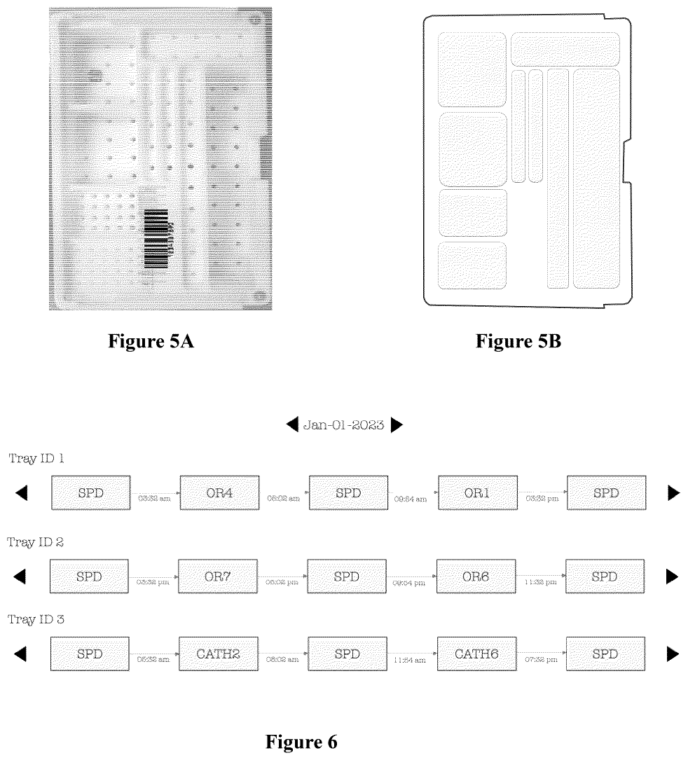

As shown in FIGS. 5A and 5B, based on a photographic image of the surgical tray, a digital template is created. FIG. 5A shows a photo of an empty tray, and FIG. 5B is a digital template of the tray created based on the photo shown in FIG. 5A using the system and method of the present invention. As shown in FIG. 5B, the digital template represents the physical structure of the tray, including the overall physical shape and dimensions of the tray and sections, regions, slots, and sub-slots within the tray.

Further, the system comprises a set of computer vision algorithms that have a mechanism to calculate the dimensions of the tray using a reference object as a calibration. A reference object is an object of known dimension. For example, a US quarter coin ($0.25) may be used as a reference object, while a photograph of the tray with the reference object next to it is input into the system. The system of the present invention uses this image to calculate the dimensions of the tray. These dimensions are used to create a wireframe template of the tray.

The digital tray template can be organized into sections and sub-sections similar to a physical tray as shown in FIGS. 2A to 3B. Each section or subsection can be associated with a specific device identifier. This device identifier can be a GUDID or a vendor specific device id or a hospital specified identifier. The Global Unique Device Identification Database (GUDID) is a database administered by the FDA that serves as a reference catalog for every device with a unique device identifier (UDI).

The system of the present invention identifies and associates a physical tray to a digital template. The system maintains a database of tray templates organized by vendors. In addition, a hospital can choose the subset of tray's they use in their facility using the configurations in the system. Hospitals can also create their own templates. A user can use a scanning mechanism to choose a template for a physical Tray. Vendor owned or Hospital owned trays are usually identified using a barcode. In absence of a bar code, the user can also use the search template function by taking the photograph of the tray. The system uses image compare algorithms to identify a set of templates that the user can pick from.

The system of the present invention records the handover of the tray between users and departments and captures the photographic and digital representation of a tray during the lifecycle. In a typical handover scenario, prefilled trays are handed over by vendors to the SPD. Once tray and medical assets loaded thereon are sterilized, they are handed over to clinical care areas like Operating Rooms to be used for a particular procedure. An alternative the flow is where the SPD fills the trays with the inventory they have. In the case, the handover is between the SPD and clinical care areas. The reverse logistics is, once the tray and its medical assets loaded thereon are utilized in a procedure or a care event, the tray and any remaining content is sent back to the SPD.

The system of the present invention provides a mechanism to capture medical assets loaded on the tray in a digital format along with photograph of the tray between the handovers. The system enables accurate calculation of what medical assets loaded thereon were used and if any medical assets loaded thereon were lost during the handovers. The system of the present invention has capability to capture digital signatures from the users involved in handover. The digital signatures might be in form of system login, fingerprint, or facial recognition.

The system of the present invention further provides a user interface to manually add and remove medical assets to a digital template. A tray can be assembled by either a vendor, typically by a medical device representative or in an SPD by an SPD user. The system of the present invention can be used by a vendor user to create a digital representation of the tray. In a typical workflow, the vendor user starts off with a tray that is completely empty or partly filled. Using the available inventory; user fills the tray with appropriate medical assets loaded thereon into the tray. During the process, the user also updates the digital tray with the exact representation of the medical assets loaded thereon. This can be manually done by using the user interface of the digital tray and updating the tray content or by scanning the tray to create visual image. Similarly, the SPD user can use the system to create a digital representation of the tray either manually or by scanning the tray once the required content is placed into the tray.

The system of the present invention uses computer vision mechanisms like Mean Squared Error algorithm, Structural Similarity Index, Histogram Analysis or Feature Extraction and matching to identify a difference between images of tray taken during the multiple handovers over the lifecycle. The functionality is used to identify the medical assets loaded thereon that were utilized between the handovers. Based on the image analysis and differences identified in various sections of the tray, the system identifies medical assets loaded thereon that were used though the section or/subsection and content ID association.

The system of the present invention utilizes computer vision and automation to capture while a user manually loads the surgical tray to identify the assets placed on the tray. A computer application that can be run on computer or tablet or a mobile device and can be placed above a tray assembly station to capture the tray information. The computer application, at the beginning of the user workflow of loading the medical assets onto a tray, take the initial reference snapshot of the tray. The computer application analyzes users hand gestures and movements to evaluate changes to the tray, for example, addition of assets to the tray or remove of assets from the tray. Based on the analysis, the computer application takes a snapshot (picture) of the tray. Further, the computer application can utilize a preconfigured timer to take a snapshot of the tray. Moreover, the computer application can be triggered to take a snapshot of the tray using specific points in the workflow, for example, when user scans the barcode of a package containing various assets. The computer application arranges pictures of the tray, beginning with the initial snapshot of picture taken at the initial stage and all subsequent snapshots taken in time sequence and sends them to the custom developed computer vision algorithm.

The computer vision algorithm, using methods such as image registration, Mean Squared Error algorithm, Structural Similarity Index, Histogram Analysis and Feature Extraction identifies the assets added to the tray by finding the difference between the images arranged in time sequence. The computer application utilizes information from the tray configuration to identify the assets added on to the tray.

As shown in FIG. 6, the system and method of the present invention have mechanism to visually represent the history of flow of tray or container across multiple users and departments. This enables administrators to track and assess the asset utilization and adherence to hospitals guidance on asset tracking.

The system and method of the present invention uses custom computer vision algorithms to identify the disposition of an asset using indicators on the tray. When a surgical tray is utilized in a clinical department, for example an operating room, assets, or parts available in the tray are removed from the tray and are used for the procedure being performed in the clinical department. These assets can be implanted into a patient body (e.g., pacemaker), used during the procedure (e.g., consumables like sponge), wasted (e.g., accidentally dropping a pedicle screw on the floor), in-and-out” (e.g., implantable assets that are used as place-holder during surgery and later removed before closure of the procedure).

The above dispositions of the assets need to be recorded in the patient records. A user can use a marker to indicate the disposition of particular asset. As an example, user write a circle around the asset slot using a colored marker. Further the color of the marker can be used as an indicator for additional details on the disposition. For example, a red marker is used to mark wasted assets. The computer application of the system of the present invention utilizes computer vision to identify such indicators and the respective disposition is associated with the asset utilization records.

Further, a user can use a fiduciary indicator, like a colored sticker dot or a colored rubber string and place them in the slot to indicate the usage. For example, in a vertically stacked tray, fiduciary indicators can be used to indicate the number of assets used and color could indicate if disposition of the asset. The computer application in the system of the present invention utilizes computer vision algorithms to identify the fiduciary indicators and associates the asset utilization and disposition based in the system configuration.

The system of the present invention uses tray configuration to identify if the user misplaces an item in a wrong location. Using the tray configuration details and the item scanned by the user, the system can detect any differences and alter the user using a warning screen or a system sound. The system thus prevents wrong assets being sent to the operating room and further prevents delays in treatment.

We claim:

1. A system for tracking and managing a surgical tray and medical assets loaded thereon, comprising
a user interface for capturing an image of a surgical tray and medical assets loaded thereon at one or more points of time and locations;
a first tangible non-transitory storage medium storing datasets related to the image, configuration, and medical assets loaded on the surgical tray at the one or more points of time and locations captured by the user interface;
a second tangible non-transitory storage medium comprising
a first dataset of information relating to configuration of surgical trays comprising information on dimensions, colors, and locations of medical assets on the surgical tray and corresponding device identifiers for the medical assets;
a second dataset of information on tray identifiers; and
a third dataset of information on customers being associated with the second dataset of information on the tray identifiers;
one or more non-transitory media being connected to the user interface and storing instructions that, when executed, causing one or more computing devices to perform steps comprising
receiving the image of the surgical tray and the medical assets loaded thereon from the user interface,
selecting a computer algorithm for assessing changes in physical details, locations, and the medical assets loaded on the surgical tray based on a pre-defined logic that maps specific algorithms to types of the surgical tray and types of the medical assets, and
sending the image of the surgical tray and the medical assets loaded thereon received form the user interface to the selected computer algorithm for assessment,
assessing the image and sending information on the surgical tray and the medical assets loaded thereon based on the assessment to the user interface; and
a set of computer algorithms that extract information or features from the image of the surgical tray and the medical assets loaded thereon received from the one or more non-transitory media.

2. The system of claim 1, wherein the system is installed on a computer, a tablet, or a handheld mobile device and further comprises a build-in camera or an external camera connected thereto for capturing the image.

3. The system of claim 1, wherein the set of computer algorithms are based on mean squared error algorithm, structural similarity index, histogram analysis, feature extraction and matching, image registration, or a combination thereof.

4. A method for tracking and managing the surgical tray and the medical assets loaded thereon using the system of claim 1, comprising
obtaining an image of the surgical tray through the user interface at an initial stage of the surgical tray's lifecycle to obtain an initial reference image,
selecting a digital template to be associated with the surgical tray,
capturing a series of images of the surgical tray and the medical assets loaded thereon during different stages of the lifecycle,
comparing multiple images of the surgical tray and the medical assets loaded thereon, identifying difference in the multiple images, and assessing one or more changes over the different stages of the lifecycle by computer vision analysis, wherein the multiple images comprise the series of the images captured during the different stages of the lifecycle, the initial reference image, and the digital template,
recording and updating the one or more changes over the different stages of the lifecycle in the first tangible non-transitory storage medium, and
sending an instruction to a user through the user interface based on the one or more changes,
wherein the multiple images of the surgical tray comprise images of a plurality of sections in each layer and the medical assets loaded in the sections of each layer in the surgical tray, and the one or more changes in the surgical comprise changes in the surgical tray, changes in the medical assets loaded thereon, or both.

5. The method of claim 4, wherein the digital template is created by
obtaining a photographic image of the surgical tray,
creating a wireframe of the surgical tray based on the photographic image by the computer vision analysis, wherein the computer vision analysis use edge and contouring detection to identify edges and shape of the surgical tray,
calculating dimensions of the surgical tray based on a reference object, and
organizing the plurality of sections of each layer in the surgical tray to create the digital template with corresponding sections and layers.

6. The method of claim 5, further comprising
associating one or more sections of the digital template with a device identifier, wherein the device identifier identifies a device to be fit into the corresponding sections of the surgical tray associated with the digital template.

7. The method of claim 5, further comprising
scanning the surgical tray and detecting and identifying medical assets filled in the surgical tray, and
modifying the digital template through the user interface based on the identified medical assets loaded on the surgical tray associated with the digital template.

8. The method of claim 4, wherein the digital template is selected based on a tray identifier provided on the surgical tray.

9. The method of claim 4, wherein the digital template is selected from a database comprising pre-determined digital templates.

10. The method of claim 9, further comprising
analyzing the initial reference image by the computer vision analysis,
obtaining and displaying a plurality of digital templates related to the surgical tray from the database comprising pre-determined digital templates on the user interface, and
selecting the digital template associated with the surgical tray among the plurality of the displayed digital templates by the user through the user interface.

11. The method of claim 4, further comprising
associating a digital signature of the user with the changes in the surgical tray.

12. The method of claim 4, wherein the changes in the surgical tray comprise a misplaced medical asset, and the instruction is to correct the misplaced medical asset.

13. The method of claim 4, wherein the changes in the surgical tray comprise changes in quantities of the medical assets in the surgical tray, and the instruction is to update accounting for the medical assets and billing in accordance with the changes.

14. The method of claim 4, wherein the changes in the surgical tray comprise changes in quantities of the medical assets in the surgical tray, and the instruction is to replenish the medical assets.

15. The method of claim 4, wherein the computer vision analysis are based on mean squared error algorithm, structural similarity index, histogram analysis, feature extraction and matching, image registration, or a combination thereof.

16. The method of claim 4, further comprising
detecting a triggering event for updating the one or more changes of the surgical tray in the one or more datasets stored in the first tangible non-transitory storage medium, and
updating the one or more datasets stored in the first tangible non-transitory storage medium,
wherein the triggering event is a hand gesture or movement by the user near the surgical tray, set by a pre-configured timer, or an action of taking a photo or scanning of the surgical tray by the user.

17. The method of claim 4, further comprising
arranging the multiple images of the surgical tray in the different stages of the lifecycle in a time sequence, and
displaying the one or more changes associated with the different stages of the lifecycle through the user interface,
wherein the one or more changes comprise usages and disposition of the medical assets and users associated with the one or more changes.

18. The method of claim 4, further comprising
identifying a fiduciary indicator on the surgical tray in the different stages of the lifecycle, and
recording the one or more changes associated with the fiduciary indicator.

19. The method of claim 4, wherein the medical assets comprise one or more implants.
